(19) United States
(12) Patent Application Publication
(10) Pub. No.: US 2019/0159710 A1
(43) Pub. Date: May 30, 2019

(54) BLOOD SAMPLING TOOL/DEVICE COMPRISING BLOOD SAMPLER AND BLOOD CONTAINER

(71) Applicant: Micro Blood Science Inc, Tokyo (JP)

(72) Inventors: HAJIME IWASAWA, Tokyo (JP); TOMOAKI NISHIMURA, Tokyo (JP); SHOTA NEMOTO, Tokyo (JP)

(21) Appl. No.: 16/097,563

(22) PCT Filed: May 19, 2017
(86) PCT No.: PCT/JP2017/018763

§ 371 Date: Oct. 29, 2018

(30) Foreign Application Priority Data
Aug. 17, 2016 (JP) 2016-160250

(51) Int. Cl. A61B 5/15; G01N 1/14; B01L 3/02; B01L 3/00

(52) U.S. Cl. CPC A61B 5/150343; A61B 5/150267; A61B 5/150274; A61B 5/150351; G01N 1/14; B01L 2300/161; B01L 3/565; G01N 2001/149; B01L 2400/0406; B01L 2400/088; B01L 3/021

(57) ABSTRACT

An exemplary blood sampling tool/device can be provided with which, even when a small amount of sample is collected, such amount of sample can be collected in a state suitable for a later analysis. The exemplary blood sampling tool/device can comprise, e.g., a pipette tip-shaped blood sampler having openings at an upper part and a lower part having an interior space for aspirating and retaining blood by the capillary action. This exemplary tool/device can also comprise a blood container having a hydrophilically treated inner wall and bottom, and having a protrusion provided at the bottom that is able to accommodate the blood collected by the blood sampler. A sealing lid can also be provided for sealing the blood container. With such exemplary blood sampling tool/device, when the lower opening of the blood sampler having aspirated and retained blood in the interior space is brought into contact with the inner wall or bottom of the blood container, the blood can easily transfer from the blood sampler into the blood container.

### CROSS REFERENCE TO RELATED APPLICATION(S)

This application relates to, and claims the benefit and priority from International Patent Application No. PCT/JP2017/018763 filed on May 19, 2017 that published as International Patent Publication No. WO 2018/034032 on Feb. 22, 2018, which claims the benefit and priority from Japanese Patent Application No. 2016-160250 filed on Aug. 17, 2016, the entire disclosures of which are incorporated herein by reference in their entireties.

### FIELD OF THE DISCLOSURE

The present disclosure relates to an exemplary blood sampling tool/device comprising a blood sampler and a blood container for collecting a small amount of blood.

### BACKGROUND INFORMATION

Condition changes and diseases in the living body generally appear in changes in components of body fluid, for example, blood, urine, saliva and the like as biological reactions. Then, a change in physical condition is monitored and diseases are detected, by measuring and analyzing components contained in body fluid. Particularly, blood tests measuring blood components are widely conducted as an effective way for investigating diseases and as a means for health management.

When measuring blood components, it is important to reliably collect the necessary amount of blood, particularly peripheral blood. Then, the collected blood is transferred into a blood container, and then subjected to analysis according to a predetermined purpose.

Further, a blood collection kit comprises a blood collecting tool and a blood collecting bottle has been suggested (see, e.g., Japan Unexamined Patent Application Publication No. 2006-288680). This blood collecting tool includes a cylindrical liquid absorbing unit for performing temporal liquid absorption disposed at the end and containing blood coagulation preventing liquid and a pushing unit which pushes the rear surface of the liquid absorbing unit to separate the liquid absorbing unit, while, a chemical solution is contained in the blood collecting bottle. When the blood collecting tool is pushed into the blood collecting bottle and then the pushing unit of the blood collecting tool is pushed, blood is eluted from the end of the blood collecting tool into the chemical solution. Since the collected blood is mixed in the chemical solution, however, plasma of the collected blood cannot be separated, and additionally, the amount of a specific component in the blood cannot be measured correctly.

Further, a blood collector for small amount of blood using a disposable pipette composed of a dropper tube and a flexible cap in communication has been described (see, e.g., Japan Unexamined Patent Application Publication No. 2005-017281). In this blood collector, liquid is absorbed temporarily, then, the absorbed liquid can be released by compressing or blowing a gas.

Likewise, there is a suggestion on a body fluid sampling apparatus composed of a body fluid sampler equipped with an aspirating part for aspirating body fluid, a body part having a ventilation passage for allowing a gas to pass there through for ejecting the body fluid aspirated by the aspirating part and a blocking part provided between the body part and the aspirating part; and a body fluid container capable of accommodating the body fluid that has been collected by the body fluid sampler (see, e.g., International Patent Publication WO 2015/034009). As described in this publication, the body fluid aspirated by the aspirating part of the body fluid sampler is transferred from the aspirating part into the body fluid container by pressure generated by a pressure generation part connected to the body part.

The blood test using the collected blood is performed using an automatic analyzer, but it is necessary to transfer the blood collected using the blood sampler into the blood container.

However, in the conventional sampling apparatus described herein above, whereas pressure is used to eject the aspirated body fluid, there may be a possibility of having problems such as, e.g., hemolysis due to the force applied upon release. Additionally, for the sake of convenience, it may be preferable for the vessel itself to be used to transfer blood into a blood container.

### Exemplary Solution to Problem(S)

In view of the above described issues, one of the objects of the present disclosure is to provide an exemplary blood sampling tool/device excellent in convenience, and even when a small amount of sample is collected, such small amount of sample can be collected in a state suitable for the later examination.

### SUMMARY OF EXEMPLARY EMBODIMENTS

To that end, the blood sampling tool according to an exemplary embodiment of the present disclosure can be provided which can comprise, e.g., (i) a pipette tip-shaped blood sampler having openings at an upper part and a lower part having an interior space for aspirating and retaining blood by the capillary action; (ii) a blood container having a hydrophilically treated inner wall and bottom that is able to accommodate the blood that has been collected by the blood sampler; and (iii) a sealing lid for sealing the blood container. When the lower opening of the blood sampler having aspirated and retained blood in the interior space is brought into contact with the inner wall or bottom of the blood container, the blood can be easily transferred from the blood sampler into the blood container.

According to the exemplary embodiments of the present disclosure, blood can be easily transferred from a blood sampler into the blood container without using any specially-designed discharge mechanism. Additionally, since extra force and pressure are not applied when discharging blood, the collected blood can be accommodated in the blood container without occurrence of problems, such as, e.g., hemolysis. Such blood can be suitable for a subsequent blood analysis.

The aspects described above and further aspects, features and advantages of the present disclosure may also be found in the exemplary embodiments which are described in the following with reference to the appended drawings and claims.

### BRIEF DESCRIPTION OF DRAWINGS

Further exemplary embodiments of the present disclosure are detailed in the description of the Figures, where this description shall not limit the scope of the present disclosure. The Figures show that:

FIG. 1 is a perspective front view of an exemplary configuration of a first embodiment of a blood sampling tool/device according to an exemplary embodiment of the present disclosure;

FIG. 2 is a side view of an exemplary embodiment of a procedure for collecting blood using a blood sampler of the blood sampling tool/device according to an exemplary embodiment of the present disclosure;

FIG. 3 is a side view of an exemplary embodiment of a procedure for a transfer of blood from the blood sampler into a blood container in the blood sampling tool/device that is shown in FIG. 1;

FIG. 4 is a side view of an exemplary embodiment of a procedure indicating another mode of a blood transfer from the blood sampler into the blood container in the blood sampling tool/device that is shown in FIG. 1; and

FIG. 5 is a side view of an exemplary configuration of a blood sampling tool/device according to another exemplary embodiment of the present disclosure.

Throughout the figures, the same reference numerals and characters, unless otherwise stated, are used to denote like features, elements, components or portions of the illustrated embodiments. Moreover, while the subject disclosure will now be described in detail with reference to the figures, it is done so in connection with the illustrative embodiments. It is intended that changes and modifications can be made to the described embodiments without departing from the true scope and spirit of the subject disclosure as defined by the appended claims.

### DETAILED DESCRIPTION OF EXEMPLARY EMBODIMENTS

Exemplary embodiments of the present disclosure will now be described in conjunction with exemplary methods and materials which can be used in the practice of the present disclosure.

Unless otherwise specified in the text, various technical terms and scientific terms used in present specification have the same meaning as commonly understood by one of ordinary skill in the art to which the present disclosure belongs. Additionally, any materials and methods equivalent or similar to those described in present specification can be used as well in the practice of the present disclosure.

Further, all publications and patents referenced in the present application constitute a part of present specification, for example, as referring to methods and materials and the like that can be used in the present disclosure, and are thus incorporated herein by reference in their entireties.

Hereinafter, blood sampling tools/devices according to exemplary embodiments of the present disclosure will be explained with reference to drawings.

### First Exemplary Embodiment

A first exemplary embodiment of the blood sampling tool/device according to the present disclosure can comprise, e.g., a pipette tip-shaped blood sampler having openings at an upper part and a lower part having an interior space for aspirating and retaining blood. For example, the lower opening and the upper opening can communicate in the interior space of the blood sampler, and at least a part of the interior space has a tapered shape from the lower opening toward the upper opening. The exemplary tool/device can be configured such that that when the lower opening is brought into contact with blood, the blood is aspirated into the interior space by the capillary action.

The exemplary tool/device can further comprise a blood container that can be configured to accommodate the blood that has been collected by the blood sampler. The blood container can have a space inside into which the blood sampler can be inserted, and a hydrophilically treated inner wall and bottom. Further, the exemplary tool/device can include a sealing lid for sealing the blood container. For example, the exemplary tool/device can be configured such that when the lower opening of the blood sampler having aspirated and retained blood in the interior space is brought into contact with the inner wall or bottom of the blood container, the blood transfers from the blood sampler into the blood container.

FIG. 1 shows a perspective side view of the blood sampling tool/device according to a first exemplary embodiment of the present disclosure. The exemplary blood sampling tool/device illustrated in FIG. 1 can comprise a blood sampler 100, a blood container 200 and a sealing lid 300 for sealing the blood container.

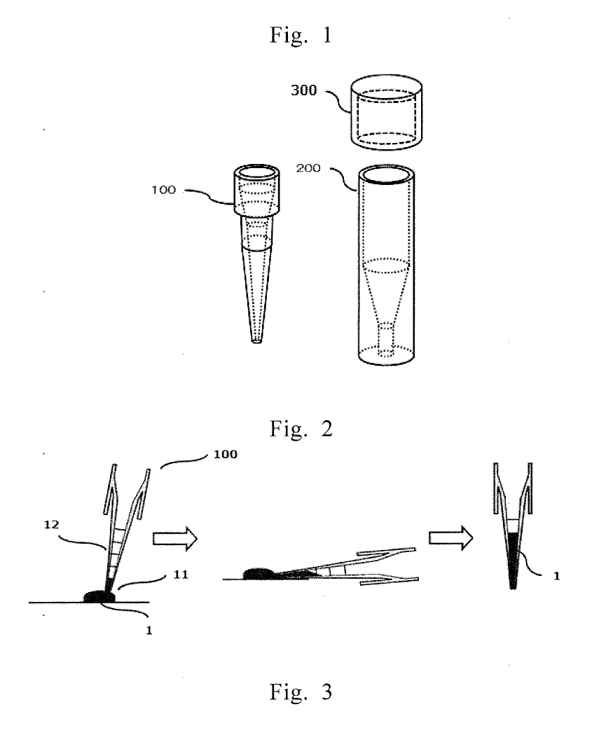

As shown in FIG. 2, when a lower opening 11 of the blood sampler 100 is brought into contact with blood 1, blood 1 is aspirated into an interior space of the blood sampler 100 by a capillary action. If the blood sampler 100 is tilted to aid in aspiration, blood 1 can be aspirated more easily. In FIG. 2, a scale 12 for checking the amount of blood is shown, although the scale 12 may or may not be attached.

For example, when collecting peripheral blood from a fingertip or an earlobe, blood can be collected by puncturing a fingertip or an earlobe and then bringing the lower opening of the blood sampler into contact with the blood that has come out. In this manner, the blood can be introduced into the blood sampler by the capillary action or a negative pressure generated by flow of fluid.

In collection of blood, e.g., there is no limit to the blood collection amount, and the blood amount can be varied according to the subsequent purpose. With the exemplary blood sampler, any small amount of blood (for example, and not by any way of limitation, any amount from 10 μL up to 200 μL) can be collected.

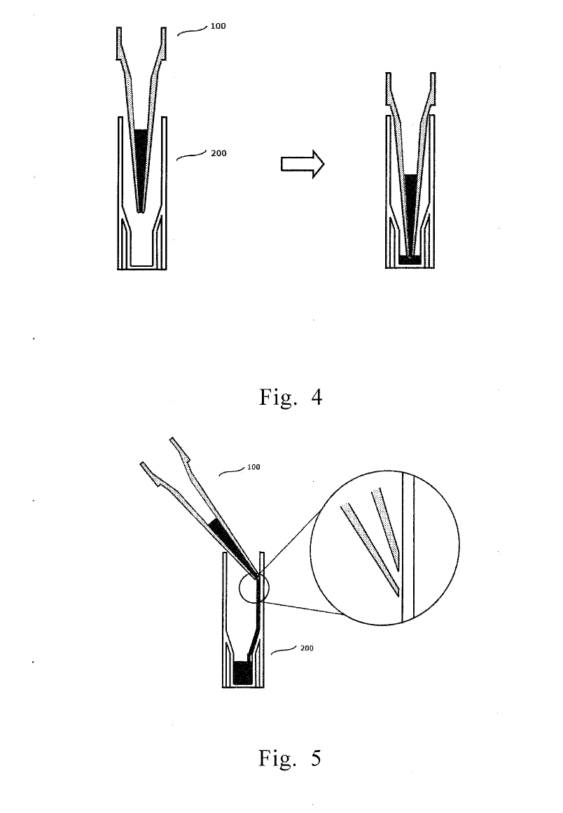

FIG. 3 illustrates a side view of the exemplary blood sampler 100 that retains blood, with the sampler 100 being inserted into the blood container 200, for transferring the aspirated blood into the blood container 200. As shown on a right side of FIG. 3, when the lower opening of the blood sampler 100 is brought into contact with the bottom of the hydrophilically treated blood container 200, the blood transfers easily into the blood container 200.

As shown in FIG. 4, the lower opening of the blood sampler 100 retaining the blood aspirated can be brought into contact with the hydrophilically treated side wall of the exemplary blood container 200. Even in such cases, blood is easily transferred from the blood sampler into the blood container by gravity and/or hydrophilic action. As shown in an enlarged right portion of FIG. 4, it is also possible to process the opening of the tip end of the blood sampler 100 obliquely.

With the blood sampling tool according to an exemplary embodiment of the present disclosure, after collecting blood using the blood sampler, the blood transfers from the blood sampler into the blood container easily by bringing the lower opening of the blood sampler into contact with the inner wall or bottom of the blood container. Before bringing the lower opening of the blood sampler into contact with the inner wall or bottom of the blood container, the blood aspirated by the capillary action can be retained in the blood sampler. If the lower opening of the blood sampler is brought into contact with the hydrophilically treated inner wall or bottom, the blood can be drawn by gravity and hydrophilic action, and the blood transfers into the blood container. For example, when the lower opening of the blood sampler in a state that blood is retained in the blood retaining portion is brought into contact with the inner wall or bottom of the blood container, the blood aspirating force (X) based on the capillary action of the blood sampler and the blood discharging force (Y) based on the gravity of the blood and the hydrophilic treatment of the blood container satisfy the relation: X<Y, thereby the blood transfers from the blood sampler into the blood container. When the inner wall of the blood retaining portion of the blood sampler is hydrophilically treated to facilitate collection of blood, movement of blood into the blood container is insufficient only by gravity and the hydrophilic treatment of the inner wall and bottom of the blood container is essential.

When the blood is transferred into the blood container using the exemplary tool/device according to an exemplary embodiment of the present disclosure, force such as, e.g., air pressure or the like for discharging the blood in the blood sampler, may be unnecessary and there is no concern of causing problems such as, e.g., hemolysis and the like.

### Second Exemplary Embodiment

A second exemplary embodiment of the blood sampling tool/device according to the present disclosure can be provided, which can be similar to the first exemplary embodiment described herein above, and in which a protrusion can be formed on the bottom of the blood container. An exemplary transfer of the blood from the blood sampler into the blood container using the blood sampling tool/device of the second exemplary embodiment of the present disclosure is shown in FIG. 5. Since the bottom (including a protrusion 21) is hydrophilically treated, when the blood in the vicinity of the lower opening of the blood sampler 100 is brought into contact with the protrusion, the blood is discharged and easily transferred into the blood container 200.

With the blood sampling tool/device according to the exemplary embodiments of the present disclosure, after blood has been transferred into the blood container, the blood container can be sealed by the sealing lid. In such exemplary state, transport and storage of the blood is possible. In addition, it is also possible to centrifuge blood in a state where the blood is accommodated in the blood container. Thus, the blood can be separated into the supernatant (e.g., serum or plasma) and the deposit (e.g., blood cell).

The hydrophilic treatment of the inner wall and the bottom of the blood container can be carried out by a conventional procedure, and can be attained by, for example, but not limited to, etching, fluorine treatment, formation of irregularities and/or fine scratches on the surface, etc. For example, it is possible to impart a hydrophilicity by forming porous parts on the inner wall surface by chemical treatments such as chemical vapor deposition, and/or by introducing a hydrophilic functional group on the inner wall surface via a catalyst. Exemplary treatments for imparting hydrophilicity to the surface of a material composed of a polymer compound, for example, polypropylene or polycarbonate are known and these procedures can be used. For example, a procedure using a mixed gas of a fluorine gas and an oxygen gas and a technology described in Hidehiko Enomoto, Toshiya Murata, “Hyomen Gijyutsu” (Surface technology), Vol. 59, No. 5, p. 282-287 (2008), which can be used without limitation thereto.

A portion for retaining blood after collection of the blood sampler 100 of the exemplary embodiments of the present disclosure (which is referred to as “blood retaining portion” in the present specification) may have a similar shape of a sample aspirating portion of a pipette tip whose inner diameter can be continuously increased. The capacity of the blood retaining portion can be preferably 200 μL or less, more preferably 150 μL or less, and even more preferably 100 μL or less.

The inner diameter of the lower end opening of the blood sampler 100 and the inner diameter of the fuselage part which is the blood retaining portion can be arbitrarily determined as long as the effect of the capillary action can be obtained. The inner diameter of the lower end opening of the blood sampler 100 can be, but not limited to, e.g., 0.1 mm or more and less than 2.0 mm, preferably 0.3 mm or more and less than 1.5 mm, and more preferably 0.5 mm or more and less than 1.0 mm, further preferably 0.7 mm or more and less than 0.9 mm. This makes it easier to collect blood from the lower opening by the capillary action.

The blood sampler can be composed of a polymer material, such as, e.g., polyvinyl chloride, polyethylene, polypropylene, acrylonitrile butadiene styrene (ABS), polycarbonate, polyethylene terephthalate and the like, from the standpoint of workability and operability.

The above exemplary materials are water repellent. Therefore, e.g., in the tip of a micropipette intending discharge after aspirating a solution, since it has separate power for aspirating and discharging, the hydrophobicity of the surface of the chip material causes high drainage when discharging the aspirated solution. When using the capillary action to aspirate blood, this property may not be preferred, thus, in the blood sampler in the exemplary tool/device according to the exemplary embodiments of the present disclosure, the inner wall of the blood retaining portion can be hydrophilically treated. When the inner wall is not hydrophilically treated, a problem may occur that blood is less likely to flow into smoothly when collecting the blood. In the blood sampler according to the exemplary embodiments of the present disclosure, the capillary action and the effect of the hydrophilic treatment of the inner wall can be combined to facilitate an easy collection of a target amount of the blood.

The hydrophilic treatment of the inner wall and the bottom of the blood container can be carried out by conventional procedures, and can be attained by, for example, but not limited to, etching, fluorine treatment, formation of irregularities and/or fine scratches on the surface, etc. For example, it is possible to impart hydrophilicity by forming porous parts on the inner wall surface by chemical treatments such as chemical vapor deposition, or by introducing a hydrophilic functional group on the inner wall surface via a catalyst. Exemplary treatments for imparting hydrophilicity to the surface of a material composed of a polymer compound, for example, polypropylene or polycarbonate are known and these methods can be used. For example, a method using a mixed gas of a fluorine gas and an oxygen gas and a technology described in Hidehiko Enomoto, Toshiya Murata, “Hyomen Gijyutsu” (Surface technology), Vol. 59, No. 5, p. 282-287 (2008) can be used without limitation thereto.

The exemplary blood container 200 is not limited in its shape and/or material so as to facilitate the blood sampler 100 to be inserted and its lower opening can be brought into contact with the wall surface or bottom of the container 200. Considering the sample collection by the operator, graspability, transportation, etc., a hard material which has high transparency can be beneficial. The sealing lid 300 of the exemplary blood container 200 is not limited in its shape and its material as long as the lid can seal the container, and it is preferable that the sealing lid is capable of preventing drying of the internal sample and elution at the time of centrifugation.

The amount of blood collected by the blood sampler 100 can be modified according to an intended examination and measurement, and the length and/or diameter of the sampler can be adjusted accordingly. The amount of the sample to be collected can be adjusted within a range of, for example, but not limited to, 10 μL to 300 μL, preferably 10 μL to 200 μL, more preferably 50 μL to 100 μL.

Further, by providing the blood sampler 100 with the guide line 12 indicating that a specific amount has been collected, it is possible to accurately collect a specific amount of blood.

By using the blood sampling tool/device according to the exemplary embodiments of the present disclosure, it is possible to prepare good blood suitable for blood analysis even with a small amount of peripheral blood collected from a fingertip or an earlobe. Such blood, by being diluted, can be analyzed according to conventional protocols and existing analyzers used for the analysis of brachial vein blood collected by a conventional blood collection tube or the like. That is, using a small amount of a peripheral blood collected from the fingertip, ear lobe or the like, the same blood analysis as before can be performed.

For example, the blood collected using the blood sampling tool/device according to the exemplary embodiments of the present disclosure can be used in examinations of general biochemical items and/or immune items, so-called blood biochemical examinations, commonly used in blood examinations, and additionally, also for special examination items such as, e.g., cancer markers and/or allergy.

### Examples of Hydrophilic Treatment of Blood Container

### Processing Example 1: Hydrophilic Treatment of Inner Wall and Bottom of Sampling Tool by Etching

A molded blood container of ABS was immersed in a solution containing hexavalent chromium for 12 minutes thereby dissolving butadiene on the surface and making etching holes on the surface, to form fine porous parts.

### Processing Example 2: Hydrophilic Treatment of Inner Wall and Bottom of Blood Container with Mixed Gas of Fluorine Gas and Oxygen Gas

In a molded blood container of polycarbonate, a hydrophilic treatment by a fluorine gas treatment was provided by Takamatsu Teisan Corporation (Takamatsu, Kagawa Prefecture, Japan) based on disclosure of Japanese Unexamined Patent Application Publication No. 2010-150460, and a surface modification treatment was conducted using a mixed gas of a fluorine gas and an oxygen gas at a fluorine gas partial pressure of 1.33 Pa and an oxygen gas partial pressure of 93100 Pa at a treatment temperature of 25° C. for a treatment time of 600 seconds.

In this fluorine treatment, hydrophilicity of a carboxyl group, a hydroxyl group or the like can be developed by utilizing high reactivity of a fluorine gas and oxygen.

### Processing Example 3: Hydrophilic Treatment of Inner Wall and Bottom of Blood Container by Surfactant Coating

The following was performed using the blood sampling tool/device according to the exemplary embodiment of the present disclosure shown in FIG. 5. In a molded blood container of polycarbonate and having a protrusion, the inner wall and the bottom were immersed in a 0.2% (w/w) polyoxyethylene sorbitan monolaurate aqueous solution for 30 seconds, then, washed with pure water and dried well, thereby performing a hydrophilic treatment by surfactant coating. When the tip of the blood sampler having aspirated 50 □L of the colored solution was brought into contact with the protrusion of the blood container, the solution moved immediately into the blood container. In contrast, the same procedure was conducted on the blood sampler not subjected to the above-described hydrophilic treatment, but the solution did not move.

The above exemplary description merely illustrates the purpose and objects of the present disclosure and is not intended to limit the scope of the appended claims. Without departing from the scope of the appended claims, various alterations and substitutions for the described embodiments will be apparent to those skilled in the art from the teachings described in the present specification.

### EXEMPLARY DESCRIPTION OF NUMERALS

1. blood
11. lower opening
12. guide line
21. protrusion
100. blood sampler
200. blood container
300. sealing lid

1-5. (canceled)

6. A blood sampling device, comprising;
a pipette tip-shaped blood sampler having upper and lower openings at an upper part and a lower part thereof, respectively, the blood sampler having an interior space that is configured and sized to aspirate and retain blood, wherein the lower opening and the upper opening communicate with one another in the interior space of the blood sampler, wherein a lower portion of the interior space defines a blood retaining portion having a first hydrophilically-treated inner wall, wherein the lower portion has a tapered shape extending from a tip of the lower opening toward the upper opening, and wherein, when the lower opening is brought into contact with blood, the blood is caused by the blood sampler to be aspirated into the interior space by a capillary action;
a blood container configured and structured to accommodate the blood collected by the blood sampler, wherein the blood container includes a space therein into which the blood sampler is insertable, and has a second hydrophilically-treated inner wall and a bottom;
a sealing lid configured to seal the blood container; and
a protrusion provided at the bottom of the blood container, wherein, when the blood retained in the blood retaining portion is brought into contact with the protrusion, the blood sampling device is configured to discharge the blood into the blood container.

7. The blood sampling device according to claim 6, wherein at least one the blood sampler or the blood container is subjected to a hydrophilic treatment by etching or fluorine processing.

8. The blood sampling device according to claim 6, wherein the blood container is subjected to a hydrophilic treatment by forming irregularities or small scratches on a surface of the inner wall or the bottom of the blood container.

9. The blood sampling device according to claim 6, wherein the lower opening is an opening having an inner diameter of between 0.5 mm and 1.0 mm.

10. A blood sampling device comprising;
a pipette tip-shaped blood sampler having upper and lower openings at an upper part and a lower part thereof, respectively, the blood sampler having an interior space that is configured and sized to aspirate and retain blood, wherein the lower opening and the upper opening communicate with one another in the interior space of the blood sampler, wherein a lower portion of the interior space defines a blood retaining portion having a first hydrophilically-treated inner wall, wherein the lower portion has a tapered shape extending from a tip of the lower opening toward the upper opening, and wherein, when the lower opening is brought into contact with blood, the blood is caused by the blood sampler to be aspirated into the interior space by a capillary action;
a blood container configured and structured to accommodate the blood collected by the blood sampler, wherein the blood container includes a space therein into which the blood sampler is insertable, and has a second hydrophilically-treated inner wall and a bottom;
a sealing lid configured to seal the blood container, wherein, when the lower opening of the blood sampler is provided in a state such that the blood is retained in the blood retaining portion is brought into contact with the inner wall or the bottom of the blood container, a blood aspirating force (X) based on the capillary action of the blood sampler and a blood discharging force (Y) based on a gravity of the blood and a hydrophilic treatment of the blood container satisfy the following relation: X<Y: and
a protrusion provided at the bottom of the blood container, wherein, when the blood retained in the blood retaining portion is brought into contact with the protrusion, the blood sampling device is configured to discharge the blood into the blood container

11. The blood sampling device according to claim 10, wherein at least one the blood sampler or the blood container is subjected to a hydrophilic treatment by etching or fluorine processing.

12. The blood sampling device according to claim 10, wherein the blood container is subjected to a hydrophilic treatment by forming irregularities or small scratches on a surface of the inner wall or the bottom of the blood container.

13. The blood sampling device according to claim 10, wherein the lower opening is an opening having an inner diameter of between 0.5 mm and 1.0 mm.
